(19) United States
(12) Patent Application Publication
(10) Pub. No.: US 2007/0191746 A1
(43) Pub. Date: Aug. 16, 2007

(54) SHOULDER DISTRACTION SPLINT

(76) Inventors: Darryl E. Barnes, Rochester, MN (US); Edward L. Schwarz, Minneapolis, MN (US)

Correspondence Address: NAWROCKI, ROONEY & SIVERTSON;SUITE 401, BROADWAY PLACE EAST, 3433 BROADWAY STREET NORTHEAST, MINNEAPOLIS, MN, 554133009, US

(21) Appl. No.: 11/552,869

(22) Filed: Oct. 25, 2006

Related U.S. Application Data
(60) Provisional application No. 60/730,495, filed on Oct. 26, 2005.

(51) Int. Cl. A61F 5/00

(52) U.S. Cl. 602/4

(57) ABSTRACT

An arm harness for applying at least one of distractive and abductive force to a human shoulder has at least two restrictor features mounted on a torso strap to be placed around a patient's waist. A first restrictor feature serves as a fulcrum at a restriction point on the arm spaced from the elbow to limit movement of the arm in one direction at the restriction point. The second restrictor feature applies force to the arm spaced both from the restriction point and from the elbow. In one embodiment, the first restrictor feature is positioned to limit movement of the forearm toward the shoulder, and the second restrictor feature applies force to the wrist to urge the wrist to move toward the shoulder, thereby applying axial distractive force to the shoulder joint. In another embodiment, the first restrictor feature is placed between the upper arm and the torso. The second restrictor feature urges the elbow toward the torso, thereby applying lateral distractive force to the shoulder joint. The two types of restrictor feature pairs may be combined in an arm harness that provides both axial and lateral distraction to the shoulder joint.

### CROSS-REFERENCE TO RELATED APPLICATIONS

This is a regular application filed under 35 U.S.C. § 111(a) claiming priority, under 35 U.S.C. § 119(e)(1), of provisional application Ser. No. 60/730,495, previously filed Oct. 26, 2005 under 35 U.S.C. § 111(b).

### BACKGROUND OF THE INVENTION

The shoulder is a ball-and-socket joint formed from a sphere-shaped convex surface on the humerus, the humeral head and a complementary concave socket surface on the scapula, the glenoid. The rotator cuff tendons and a soft tissue capsule hold the two surfaces in the proper approximation in relationship to each other. When functioning properly, a shoulder provides full, pain-free range of motion through 180 degrees of abduction, 45 degrees of adduction, 90 degrees of flexion, 45 degrees of external rotation and 55 degrees of internal rotation.

This normal function is often lost because of injury, overuse, or excessive stress on the shoulder joint. The result is rotator cuff tendonitis and subacromial impingement (contact) that accounts for nearly one-third of all shoulder complaints evaluated in clinics. Injury that leads to inflammation and/or pain and weakness of the rotator cuff tendons that hold the shoulder joint together often causes impingement of the subacromial structures between the acromion (bone) and the humeral head (bone), primarily the rotator cuff, resulting in painful symptoms.

FIG. 7 shows the physiology involved. The upper arm bone, the humerus, identified as UA in FIG. 7, terminates in the humeral head 60 at the upper end of the humerus. The acromion 63 is positioned directly above the humeral head 60. A person with normal physiology has a large impingement interval, or spacing, 68 between the acromion 63 and the humeral head 60.

For any number of reasons, the humeral head 60 may be pulled toward the acromion 63 as shown by the phantom view of a humeral head so displaced as at 60a. This displacement of humeral head 60 reduces the impingement interval 68 to that shown at 68a. This reduced impingement interval 68a can cause pain, range of motion deficit, and stiffness in the shoulder joint.

This problem has substantial adverse impact on those who suffer from it. Range of motion is affected and pain occurs to the point that many normal activities are difficult. The pain is often to a level that interferes with sleep, which has further potentially serious consequences for the patient.

A number of conventional treatments exist for this condition. Physical therapy is often successful over time. However, this treatment often requires frequent visits to the therapist, which is time-consuming and expensive. Similarly, the patient can do specific exercises that are successful over time, but again may be painful, require discipline, and are time-consuming. Because these treatments have little likelihood of undesirable side-effects, they are a preferred way to treat.

Medications such as NSAIDs and prescription palliatives are often successful. However, NSAIDs often do not provide adequate relief or may be contraindicated in some patients. The same is true for prescription drugs, which may be habit-forming as well. Both classes of drugs have well-known undesirable side effects such as GI problems, renal problems, drowsiness, dry mouth, etc.

Surgery is another possibility, but is both expensive and potentially risky, and doesn't necessarily even solve the underlying problem. Surgical replacement of the joint is also possible, but reduces shoulder functionality and strength; is expensive; is painful, risky, and costly, and does not always solve the problem.

Splints and gentle, passive traction devices may also provide relief and may even be curative. However, there is no current night time splint specifically for relief of subacromial impingement when the patient is prone or sleeping.

The problem is particularly acute at night when the patient is attempting to sleep. The prone or supine position or lying on one's side applies forces on the shoulder that are different from those in the standing position. These different forces often cause discomfort or pain for the patient.

### BRIEF DESCRIPTION OF THE INVENTION

This invention is a device in the nature of a splint or harness designed to provide an adjunct to current medical therapy which includes: relative rest, anti-inflammatory medications (NSAIDs, corticosteroid injection) and physical therapy. The harness is particularly suitable for wear during nighttime rest when the patient is prone, supine or lying on one side in a bed.

A number of definitions are important in understanding the invention. The first is the meaning of “distraction”, the term for force applied along the length of the humerus and away from the shoulder. The second is “abduction”, the term for force tending to pull the elbow outwardly and away from the torso. A third useful definition is for “anticubital”, which means the anterior portion of the elbow joint. A fourth useful definition is for “impingement interval” which is the distance between the most superior part of the shoulder (the under surface of the acromion) and the most superior portion of the humeral head. “Axial” with respect to the upper arm refers to force or motion along the humerus. “Lateral” refers to movement or force of the humeral head away from the scapula.

The splint design in accordance with the present invention provides at least one of gentle axial and lateral distraction using passive forces to relieve shoulder pain such as may occur nocturnally or otherwise caused by abnormalities in the shoulder, including rotator cuff tendonitis and subacromial impingement.

The splint consists of a wide torso strap with specialized restrictor features mounted in certain positions thereon that when worn by a patient, cooperate to position the patient's arm in a preferred position and with certain types of force applied to the humerus.

One of these features is a pad that is adjustable to the size of the patient to increase or decrease abduction and provide a well-padded fulcrum (anticubital component).

A strap that attaches the wrist to the abduction pillow or to the torso strap by Velcro or similar attachment provides the gentle distraction force to the shoulder joint by way of a fulcrum effect. When the wrist strap is placed in a more cephalad position, there will be more distraction force passively placed on the shoulder, thereby increasing or stabilizing the impingement interval and thus decreasing impingement. The splint also provides good shoulder joint stabilization during sleep, preventing uncomfortable positioning which may result in poor sleep.

Materials from which the strap is made may include, but are not limited to, cloth and other types of permeable and flexible sheeting. Velcro is useful for the various types of fasteners required. The pads may be made from foam, rigid plastic, and if adjustability is helpful, air bladders.

Such a splint may comprise an arm harness providing at least one of abduction and distraction for a human patient's shoulder joint. Such an arm harness includes a torso strap having a length and a width, and an upper and lower edge. This arm harness is intended to encircle the patient's waist with the upper edge closer to the patient's shoulder joint than is the lower edge. The strap also has front and side areas. The strap is to be fastened to the patient with the front and side areas in approximate alignment with the front and side of the patient's torso.

The torso strap supports a restrictor feature having a contact surface for contacting a preselected portion of the patient's arm at a point spaced from the patient's elbow. In a first version the restrictor feature is located at a point on the strap for contacting the patient's forearm. In a second version, the restrictor feature is located at a point on the strap contacting the inside of the patient's upper arm between the elbow and the shoulder.

The torso strap also supports an arm holder for loading a portion of the patient's arm at a point spaced from the preselected arm portion where the restrictor feature contacts the patient's arm. “Loading” in this context means applying gentle force to the arm to urge the part of the arm to which the loading is applied, closer to the torso strap. The loading is transverse to the length of the arm portion involved. When the patient's arm is so loaded, by pressing the preselected arm portion against the contact surface, the shoulder joint is subjected to force tending to gently pull the humeral head back toward neutral alignment with the glenoid achieving normalization/stabilization of the impingement interval.

In the first version, the restrictor feature has a contact surface facing toward the lower edge of the strap and is positioned to allow the upper surface of the patient's forearm to contact the restrictor feature's contact surface. The arm holder is positioned to place the restrictor feature between the elbow and the arm holder. The restrictor feature forms a fulcrum that cooperates with the arm holder to apply gentle distractive force along the length of the humerus, tending to pull the humeral head back toward neutral alignment with the glenoid achieving normalization/stabilization of the impingement interval.

In the second version, the restrictor feature has a contact surface facing away from the surface of the strap and at the strap and is positioned to allow the upper surface of the patient's forearm to contact the restrictor feature's contact surface. The arm holder is positioned to place the restrictor feature between the elbow and the arm holder. The restrictor feature forms a fulcrum that cooperates with the arm holder to apply gentle abductive force along the length of the humerus, tending to pull the humeral head back toward neutral alignment with the glenoid achieving normalization/stabilization of the impingement interval. A preferred embodiment incorporates both the first and second versions in a single unit. Often, a patient finds superior relief from shoulder pain when the shoulder joint is subjected to both abduction and distraction forces.

### BRIEF DESCRIPTION OF THE DRAWINGS

FIGS. 1a and 1b are vertically aligned projections of the front elevation and top elevation respectively of a first version of the arm harness when laid out on a flat horizontal surface.

FIG. 1c shows the top elevation of FIG. 1b with a cross section image of a patient's upper arm positioned in the arm harness.

FIGS. 2a and 2b are vertically aligned projections of the front elevation and top elevation respectively of a second version of the arm harness when laid out on a flat horizontal surface.

FIG. 2c shows the top elevation of FIG. 2b with a cross section image of a patient's upper arm positioned and held in the arm harness.

FIGS. 3a and 3b are substantially vertically aligned projections of the front elevation and top elevation respectively of a preferred embodiment of the arm harness when laid out on a flat horizontal surface.

FIG. 3c shows the top elevation of FIG. 3b with a cross section image of a patient's upper arm positioned and held in the arm harness.

FIG. 4 shows components of the arm harness that can be assembled with a variety of dimensional parameters.

FIG. 5 is a perspective view showing the arm harness in an intermediate position as a patient is attaching the arm harness.

FIG. 6 is a perspective view showing the arm harness as it may appear when attached to a patient.

FIG. 7 is a sketch showing the physiology of the human shoulder and the deficit that may occur.

FIG. 8 is a sketch showing the forces created by the arm harness on a patient's arm.

### DESCRIPTION OF THE PREFERRED EMBODIMENTS

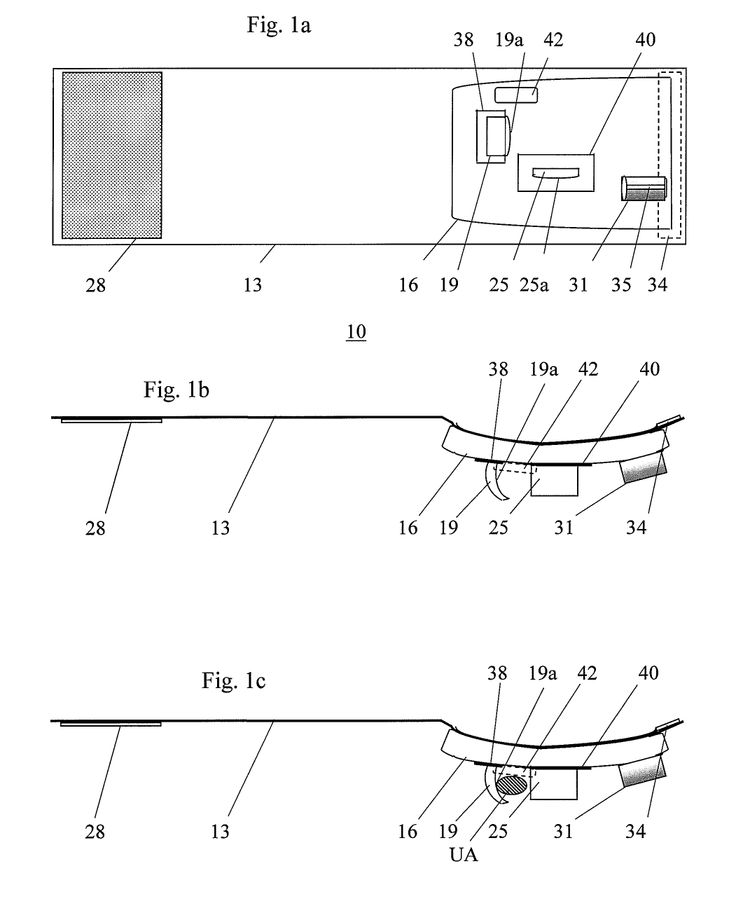

FIGS. 1a and 1b show front and top elevation projections of an arm harness 10 for providing gentle axial and lateral distraction and a variable level of abduction using passive forces to relieve shoulder pain. A patient will usually wear harness 10 at night, but as more experience is gained with a number of users, harness 10 may also have uses during other times. Harness 10 shown in FIGS. 1a and 1b is intended to support a patient's right arm. Of course, a similar harness can be provided for a left arm.

Arm harness 10 comprises a wide belt 13 and a thicker mounting base 16, which together may comprise a torso strap. Base 16 is attached to belt 13 so that base 16 can be comfortably and securely mounted on a patient's torso by wrapping belt 13 around the patient's waist and fastening the end of belt 13 to base 16. Given that typical adult male waists run from 32-40 in. (65-100 cm), base 16 and belt 13 may in combination be 44 in. (110 cm.) in the horizontal direction of FIGS. 1a and 1b. Belt 13 may be as wide as 12 in. (30 cm.) or even wider. Base 16 may be comprised of a block of flexible foam approximately 1 in. (25 mm.) thick and 16 in. (40 cm.) in the horizontal direction of FIG. 1a.

A first hook-and-loop sheet 28 such as is commonly known by the trademark Velcro is mounted on an outer surface (shown facing the viewer in FIG. 1a and on edge in FIG. 1b) of belt 13. A second, complementary hook-and-loop sheet 34 (shown in phantom in FIG. 1a and on edge in FIG. 1b) is mounted on an inner surface of belt 13 near the right end thereof. Sheet 28 may be 12 in. (30 cm.) long and sheet 34 may be 2 in. (5 cm.) long, both in the horizontal direction of FIG. 1a.

Belt 13 may comprise a variety of plastic or fabric materials, including but not limited to vinyl and heavy cotton or polyester fabric. A presently preferred belt 13 comprises 0.125 in. (0.3 cm.) thick neoprene. Further experience may lead to preferred embodiments for a belt 13. having varying thickness along either or both of the length or width (vertical dimension).

Base 16 may be formed from soft plastic or relatively rigid foam so that base 16 can comfortably mold to the patient's torso. Base 16 incorporates at least two arm movement limiters, generally referred to hereafter as either a restrictor feature or an arm holder. Each restrictor feature or arm holder reduces or limits movement of the patient's arm within at least one degree of freedom. Each restrictor feature and arm holder is attached to the outer surface of base 16 (shown facing the viewer in FIG. 1a), in a spaced relationship to be described. Each restrictor feature is associated with an arm holder. The spacings stated between the restrictor features are only general guides since the size of individual patients' arms can vary substantially. In addition, the invention contemplates means for attaching at least some of the restrictor features in a variety of positions on base 16.

In this particular nomenclature, a “degree of freedom” refers to the ability to the patient's normal ability to move the arm linearly forward and backward relative to the patient's body, linearly from side to side relative to the patient's body, rotationally about a vertical axis, or rotationally about a horizontal axis. “Reduces or limits movement” means that the patient encounters substantial resistance in attempting to move the arm in at least one direction of a degree of freedom, or may even be blocked or locked from the particular type of arm motion.

A forearm block 25 forms a restrictor feature that opposes movement in a single degree of freedom. Forearm block 25 has a downwardly facing contact surface 25a oriented generally in the horizontal plane of FIG. 1a. Contact surface 25a should be in or below horizontal alignment with the bottom edge of an elbow block 19, that is, aligned with a horizontal line intersecting the bottom edge of block 19. Block 25 may be well padded for comfort of the patient.

The edge of block 25 closer to elbow block 19 should be approximately 6-8 in. (15-20 cm.) from a vertical extension of elbow block 19. Contact surface 25a may be 4-6 in. (10-15 cm.) long. The center of contact surface 17a should align approximately with the center of a patient's forearm with the patient's elbow or upper arm pressed against elbow block contact surface 19a. Block 25 may include a mounting feature 40 that cooperates with base 16 to allow for a variety of locations and angulations of contact surface 25a on base 16.

A wrist clamp or cuff 31 forms a second restrictor feature of base 16. Cuff 31 is positioned on base 16 on the side of forearm block 25 opposite to the elbow block 19. The spacing between cuff 31 and forearm block 25 may be similar to that between elbow block 19 and forearm block 25. Cuff 31 preferably has a hook and loop closure 35 that firmly but not tightly holds the patient's wrist to prevent any substantial movement of the wrist in either rotation or translation. Cuff 31 may have a rubber-like interior surface that resists slippage of the wrist within cuff 31.

The horizontal alignment of cuff 31 with forearm block contact surface 25a is important. Generally, cuff 31 should be mounted so that the upper, inner, surface of cuff 31 is in substantial horizontal alignment with block surface 25, or perhaps slightly above this position.

An upper arm or elbow block 19 forms a part of base 16 and is a third of these restrictor features. An elbow block contact surface 19a is oriented generally vertically as shown in FIG. 1a. The purpose of block 19 is to reduce or restrict the movement of the patient's upper arm and elbow backward relative to the patient's body. In general, belt 13 mounts or attaches on the patient's body so that block 19 is positioned generally laterally or to the side of the patient's torso. Block 19 may include a mounting feature 38 that cooperates with base 16 to allow for a variety of locations and angulations of contact surface 19a on base 16. Certain designs may not require an elbow block 19.

One purpose of harness 10 is to apply a small and comfortable amount of distractive force along the axis of the humerus that tends to pull the humeral head back toward neutral alignment with the glenoid achieving normalization/stabilization of the impingement interval. Cuff 31 applies a small amount of steady upward force to the patient's wrist. Block 25 opposes the upward force applied to the patient's wrist by cuff 31, thereby forming a couple tending to rotate the patient's arm counterclockwise as shown in FIG. 1a. This couple provides downward force (distraction) on the elbow joint, which is carried by the humerus to the shoulder joint.

An abduction pad 42 on base 16 is a fourth restrictor feature, and allows for increased abduction of the humerus. Pad 42 is shown in phantom in FIG. 1b. Gravity tends to hold the arm and elbow close to the torso. Pad 42 operates as an abductor pad to abduct the humerus rotating the humeral head on the glenoid, effectively shortening the rotator cuff and decreasing traction forces on it .

In addition, the curve in contact surface 19a urges the elbow and upper arm closer to the torso when the wrist cuff 31 is attached to press the patient's elbow and upper arm firmly against the contact surface 19a, to thereby create lateral distraction. Pad 42 may be permanently mounted on base 16, or may have a mounting feature such as mounting features 38 and 40, that allows pad 42 to be mounted in positions appropriate for various patient's sizes and builds.

A typical pad 42 may be 1-2 in. (2.5-5 cm.) thick, that is the direction normal to FIG. 1a. The thickness should be sufficient to create gentle abductive force on the shoulder joint. The vertical dimension as shown in FIG. 1a may be 6-8 in. (15-20 cm.), and should be great enough so that the flow of blood in the arm area in contact with pad 42 is not affected.

FIG. 1c shows a cross section of an upper arm indicated by the UA legend. The curve in contact surface 19a gently loads the patient's elbow to urge same toward base 16. Pad 42 serves to abduct the upper arm and apply lateral distractive force on the shoulder joint.

FIG. 8 shows the physics and forces involved in axial distraction. Arm harness 10 creates force on upper arm UA away from the shoulder joint as indicated by arrow 75. This force on upper arm UA arises from force applied to forearm 65 by forearm block 25. Wrist cuff 31 prevents clockwise rotation of the forearm 65, stabilizing forearm 65 position. (FIG. 8 shows the patient's hand 78 for reference purposes.) The force applied to upper arm UA pulls humeral head 60 away from acromion 63 to create a near normal impingement interval 68. Pain and swelling is reduced, and healing may result from reduced unnatural contact between humeral head 60 and acromion 63.

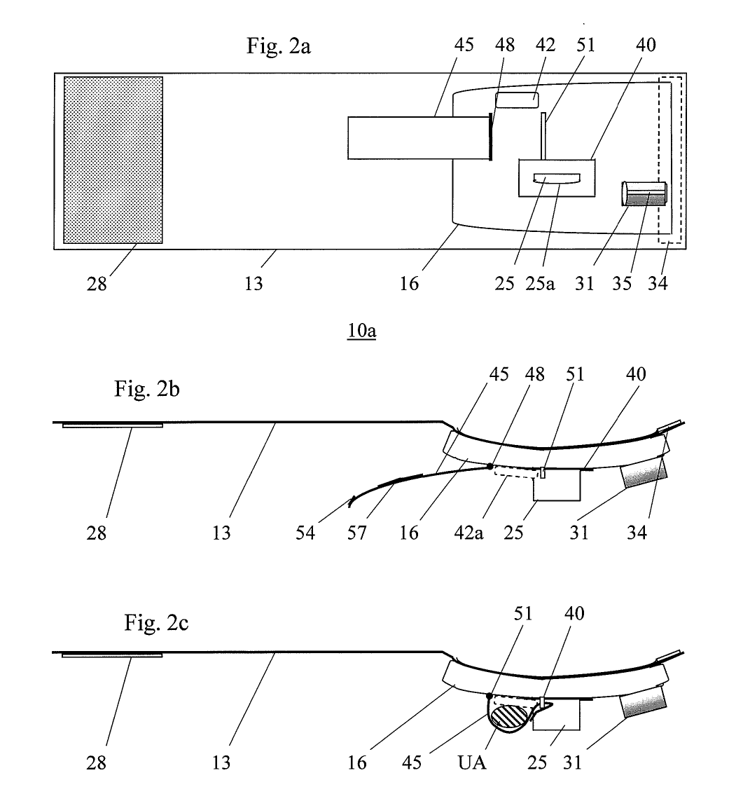

FIGS. 2a and 2b show a second arm harness 10a of the harness 10 shown in FIGS. 1a and 1b. Harness 10a has somewhat different structure for creating abduction. An elbow belt 45 replaces elbow block 19. An attachment feature 48 attaches an end of belt 45 to base 16 somewhat rearward of abduction pad 42. The length of elbow belt 45 is generally in alignment with the length dimension of belt 13.

A closure element 51 has a slot aligned generally with the width dimension of belt 13. The element 51 slot can accept the free end of elbow belt 45, allowing the free end thereof to easily slide through the element 51 slot.

Belt 45 has fastening elements on the free end of belt 45, shown as the left end of belt 45. A first hook and loop material patch 54 is attached near the free end of belt 45, on the surface facing belt 13 and away from the viewer as shown in FIGS. 2a and 2b. A second complementary patch 57 of hook and loop material is attached between patch 54 and attachment feature 48 on the surface of belt 45 facing belt 13 and away from the viewer.

A patient provides abduction force to the shoulder by slipping the end of belt 45 through the slot of element 51. By pulling on the free end of belt 45, the patient's elbow is loaded to pull the elbow close to base 16 so as to press his upper arm against pad 42. This causes gentle lateral force on the humeral head, normalizing the impingement interval between the humeral head and the glenoid surface of the scapula.

FIG. 2c shows a cross section of an upper arm indicated by the UA legend. Belt 45 gently loads the patient's elbow to urge the elbow toward base 16. Pad 42 serves as an abduction pad and block 25 serves as a fulcrum to cause gentle axial distractive and stabilization force on the shoulder joint.

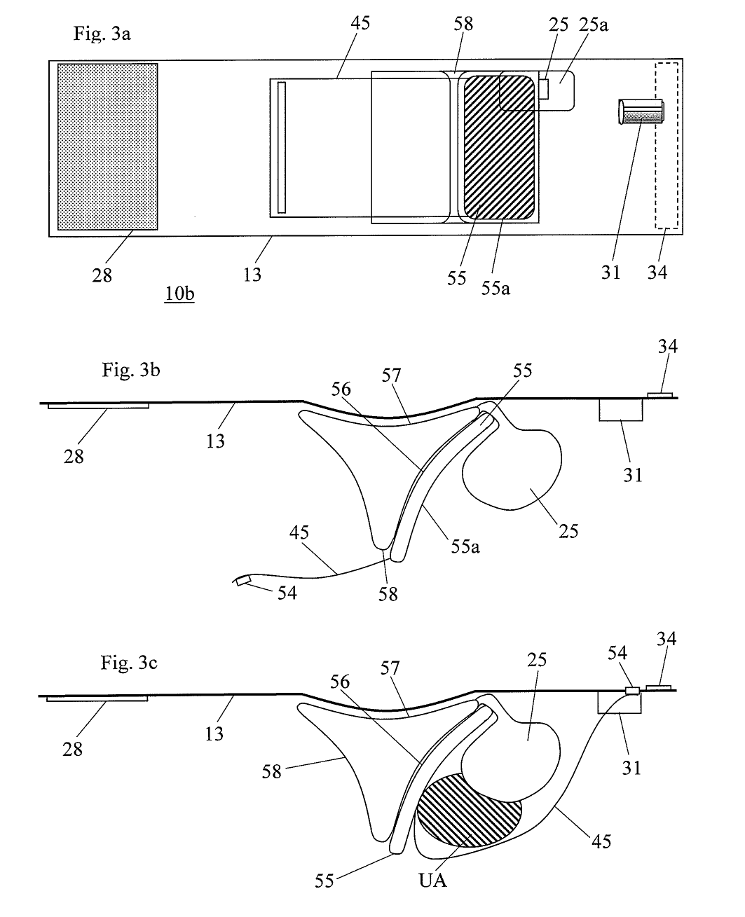

FIGS. 3a-3c show another arm harness 10c with a design that combines structure for providing both axial distractive force to the humeral head and abduction of the humerus. Reference numbers generally follow those of FIGS. 1a-1c and 2a-2c. FIGS. 3a-3c show the currently preferred embodiment.

An upper arm block or abduction wedge 58 serves as a restrictor element having a diagonally oriented pad 55 with a contact surface 55a against which the back and inner surface of the patient's upper arm presses for restricting movement of the patient's upper arm both towards the torso and rearwards. Contact surface 55a faces both outwardly and forwardly with respect to the adjacent portion of torso strap 13. In this design block 58 serves as base 16. FIG. 3c shows the upper arm of the patient as cross section UA. Belt 45 serves to secure the patient's upper arm against block 58. The design shown in FIGS. 3a-3c is a preferred embodiment for the invention.

A useful feature of block 58 is the two similar surfaces 56 and 57 that are angled with respect to each other. In FIGS. 3a-3c, pad 55 lies on surface 56, and surface 57 faces inwardly to rest against the patient's torso. The configuration of FIGS. 3a-3c is for a patient's right arm. Block 58 can be reconfigured for use with the left arm by remounting block 58 with surface 57 facing outwardly and supporting pad 55. Surface 56 then attaches to torso strap 13 and rests against the patient's torso.

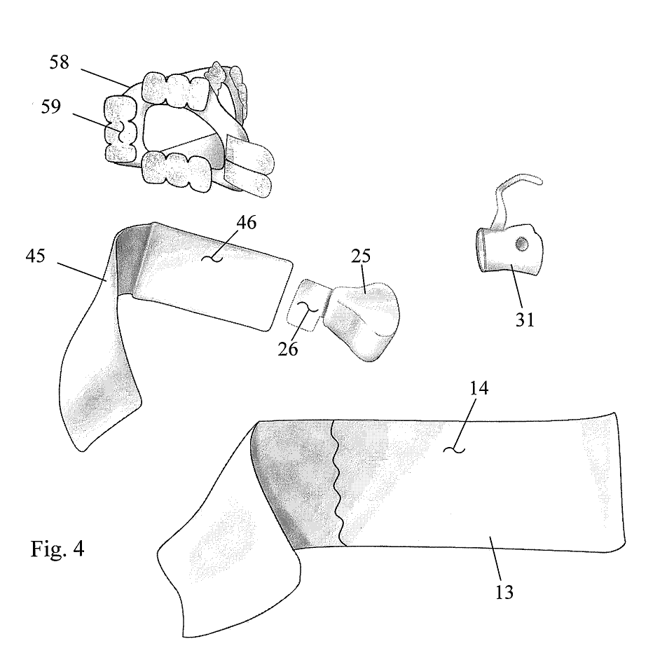

FIG. 4 shows further details of the embodiment of FIGS. 3a-3c. Individual restrictor features are separate components that can be attached at points on torso strap 13 for maximum functionality for the individual patient's needs. FIG. 4 shows upper arm block 58, belt 45, forearm block 25, and wrist cuff 31. An important advantage of FIG. 4 is the ability for repositioning the individual components to provide for use with either the left or the right arm.

Torso strap 13 has a surface 14 that comprises one part of two complementary parts of a hook and loop attachment material. Surface 14 need not overlay an entire side of strap 13, as suggested by the vertical wavy line shown. Each of the restrictor features has one part of the hook and loop material forming a part of the restrictor feature's attachment surface.

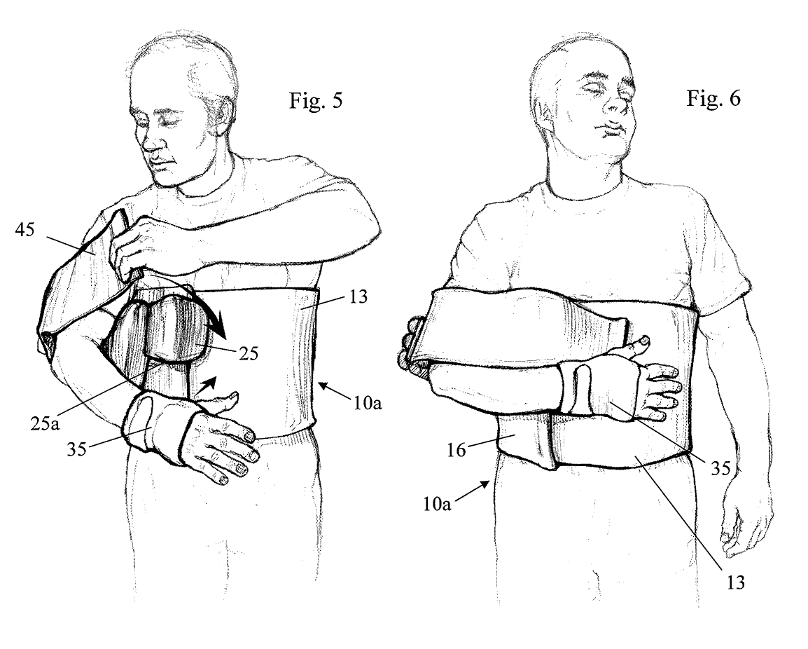

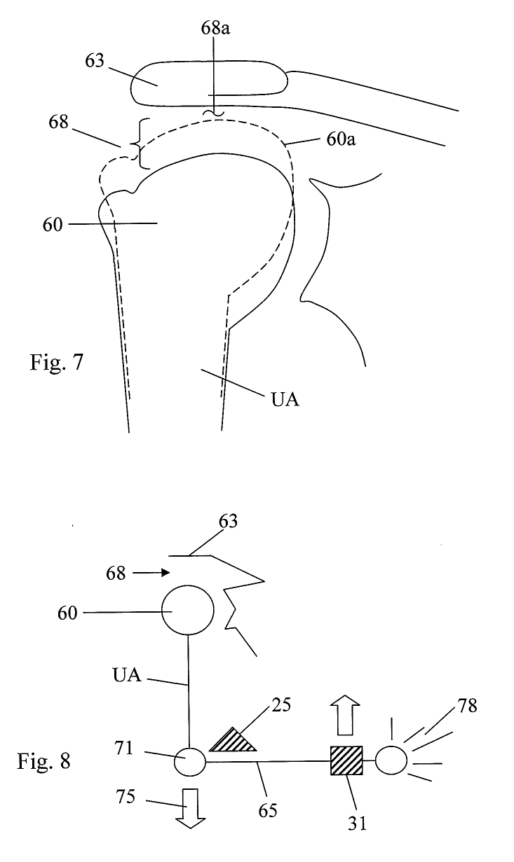

Thus, block 58 has an attachment surfaces 59, block 25 has an attachment surface 26, and wrist cuff 31 has a similar attachment surface, not shown. Elbow belt 45 is slightly different, in that the surface opposed and oppositely facing to surface 46 has an attachment surface having a hook and loop part complementary to the hook and loop material on surface 14, so that belt 45 can be attached to surface 14. Surface 46 has the part of the hook and loop material identical to that on surface 14. This allows the patient to mount block 58 at least partly on belt 45. FIG. 5 shows the arm harness 10c shown in FIGS. 3a-3c and 4 with belt 13 holding base 16 onto a patient's waist but with harness 10a not yet completely attached. FIG. 5 shows arm harness 10c attached to the patient to provide both abduction and distraction to the patient's shoulder joint.

1. An arm harness for providing at least one of lateral and axial distraction of a patient's shoulder joint, comprising:
a) a torso strap having a length and a width, and an upper and lower edge, for encircling the patient's waist with the upper edge closer to the patient's shoulder joint than is the lower edge, said strap having front and side areas, said strap to be fastened to the patient with the front and side areas in approximate alignment with the front and side of the patient's torso;
b) a restrictor feature supported on the torso strap, said restrictor feature having a contact surface for contacting a preselected portion of the patient's arm at a point spaced from the patient's elbow; and
c) an arm holder supported on the torso strap for loading a portion of the patient's arm at a point spaced from the preselected arm portion, said loading transverse to the length of the arm portion involved, and when so loading the patient's arm, pressing the preselected arm portion against the contact surface.

2. The arm harness of claim 1, wherein the restrictor feature is attached to the torso strap's front area with the contact surface in substantial alignment with the torso strap's length and facing generally away from the upper edge.

3. The arm harness of claim 2, wherein the restrictor feature comprises a wall projecting from the torso strap.

4. The arm harness of claim 3, wherein the arm holder comprises a wrist clamp spaced from the restrictor feature generally toward a part of the torso strap aligned with a vertical centerline of the torso.

5. The arm harness of claim 4, wherein the wrist clamp comprises a wrist cuff for encircling the patient's wrist.

6. The arm harness of claim 5, wherein the wrist holder comprises a cuff oriented to encircle a patient's wrist, said cuff generally aligned with the torso strap length

7. The arm harness of claim 1, wherein the restrictor feature projects from the side area of the torso strap substantially below the shoulder joint, said restrictor feature having a contact surface generally parallel to and facing away from the adjacent torso surface and toward the patient's arm; and wherein the arm holder is located on the strap between the strap's lower edge and the restrictor feature, said arm holder applying load to the arm adjacent to the elbow to urge the elbow toward the torso.

8. The arm harness of claim 7, wherein the arm holder comprises a belt attached to the torso strap at an attachment point, and a fastener spaced from the attachment point.

9. The arm harness of claim 7, wherein the restrictor feature contact surface faces outwardly and forwardly with respect to an adjacent portion of torso strap.

10. The arm harness of claim 1, including a first restrictor feature associated with a first arm holder said first restrictor feature attached to the torso strap's front area with the contact surface in substantial alignment with the torso strap's length and facing generally away from the patient's shoulder; and a second restrictor feature associated with a second arm holder, said second restrictor feature projecting from the side area of the torso strap substantially below the shoulder joint, said restrictor feature having a contact surface generally parallel to and facing away from the adjacent torso surface and toward the patient's arm.

11. The arm harness of claim 9, including a second arm holder located on the strap between the strap's lower edge and the restrictor feature, said arm holder for applying load to the arm adjacent to the elbow to urge the elbow toward the torso.

12. The arm harness of claim 10, wherein the second arm holder comprises a structure projecting from the torso strap with a concave surface adapted to receive the posterior surface of the patient's upper arm adjacent to the patient's elbow.

13. The arm harness of claim 1, including a first part of a hook and loop material covering the front and side areas of the torso strap, and wherein at least one of the restrictor feature and the arm holder has a first attachment surface, and a second part of the hook and loop material attached to the first attachment surface.

14. The arm harness of claim 13, wherein at least one of the restrictor feature and the arm holder has a second attachment surface covered by the first part of a hook and loop material.

15. The arm harness of claim 13, wherein the at least one of the restrictor feature and the arm holder first and second attachment surfaces are opposed and oppositely facing.

16. The arm harness of claim 1, wherein the restrictor feature is attached to the torso strap's front area with the contact surface in substantial alignment with the torso strap's length and facing generally away from the upper edge, and further comprising an upper arm block attached to the torso strap's side area for supporting the patient's arm in an abducted position.

17. The arm harness of claim 16, including an elbow strap securing the upper arm against the upper arm block.
